SEQUENCE LISTING (10 sequences)

SEQ ID NO: 1 - protein, 752 aa, synthetic construct
GAGQSSPATGSQNQSGNTGSIINNYYMQQYQNSMDTQLGDNATSGGSNEGSTDTTSTHTTNTQNNDWFSKLASSAFSGLFGALLADKKTEETTLLEDRILTTRNGHTTSTTQSSVGVTYGYATAEDFVSGPNTSGLETRVAQAERFFKTHLFDWVTSDPFGRCHLLELPTDHKGVYGSLTDSYAYMRNGWDVEVTAVGNQFNGGCLLVAMVPELCSIQKRELYQLTLFPHQFINPRTNMTAHITVPFVGVNRYDQYKVHKPWTLVVMVVAPLTVNSEGAPQIKVYANIAPTNVHVAGEFPSKEGIFPVACSDGYGGLVTTDPKTADPAYGKVFNPPRNMLPGRFTNFLDVAEACPTFLHFEGDVPYVTTKTDSDRVLAQFDLSLAAKHMSNTFLAGLAQYYTQYSGTINLHFMFTGPTDAKARYMIAYAPPGMEPPKTPEAAAHCIHAEWDTGLNSKFTFSIPYLSAADYAYTASDTAETTNVQGWVCLFQITHGKADGDALVVLASAGKDFELRLPVDARTQTTSAGESADPVTATVENYGGETQVQRRQHTDVSFILDRFVKVTPKDQINVLDLMQTPAHTLVGALLRTATYYFADLEVAVKHEGNLTWVPNGAPEAALDNTTNPTAYHKAPLTRLALPYTAPHRVLATVYNGNSKYGDGTVANVRGDLQVLAQKAARALPTSFNYGAIKATRVTELLYRMKRAETYCPRPLLAIHPDQARHKQKIVAPVKQLLNFDLLKLAGDVESNPG

SEQ ID NO: 2 - protein, 213 aa, synthetic construct
SGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRPIFSGEALTYKDIVVCMDGDTMPGLFAYRAATKAGYCGGAVLAKDGADTFIVGTHSAGGNGVGYCSCVSRSMLLKMKAHIDPEPHHE

SEQ ID NO: 3 - protein, 245 aa, synthetic construct
DIEPPTPICPEICSCPAAEVLGAPSVFLFPPKPKDILMISRTPKVTCVVVDVSQEEAEVQFSWYVDGVQLYTAQTRPMEEQFNSTYRVVSVLPIQHQDWLKGKEFKCKVNNKDLLSPITRTISKATGPSRVPQVYTLPPAWEELSKSKVSITCLVTGFYPPDIDVEWQSNGQQEPEGNYRTTPPQQDVDGTYFLYSKLAVDKVRWQRGDLFQCAVMHEALHNHYTQKSISKTQGKEQKLISEEDL

SEQ ID NO: 4 - DNA, 2256 bp, synthetic construct
  source 1..2256: mol_type = other DNA
ggagcaggacagagctcccctgcaaccggctctcagaaccagagcggcaatacaggcagcatcatcaacaattactatatgcagcagtatcagaactccatggacacccagctgggcgataacgccacaagcggcggctccaatgagggctccacagataccacatctacccacaccacaaatacacagaacaatgactggtttagcaagctggcctctagcgccttttccggcctgttcggcgccctgctggcagataagaagaccgaggagacaaccctgctggaggacagaatcctgaccacacgcaacggccacaccacatccaccacacagtcctctgtgggcgtgacctacggctatgcaacagcagaggacttcgtgagcggaccaaatacctccggcctggagacaagggtggcacaggcagagcggttcttcaagacccacctgtttgactgggtgacaagcgatcccttcggccggtgccacctgctggagctgcctaccgaccacaagggcgtgtacggctccctgacagattcttacgcctatatgagaaacggatgggacgtggaggtgaccgcagtgggaaaccagtttaatggaggatgcctgctggtggcaatggtgcctgagctgtgctccatccagaagcgcgagctgtatcagctgaccctgtttccccaccagttcatcaaccctcggaccaatatgacagcccacatcacagtgcctttcgtgggcgtgaacagatacgaccagtataaggtgcacaagccatggaccctggtggtcatggtggtggcccctctgacagtgaatagcgagggcgccccacagatcaaggtgtacgccaacatcgccccaaccaatgtgcacgtggccggcgagtttcctagcaaggagggcatcttcccagtggcctgctccgatggatacggaggcctggtgaccacagaccctaagaccgccgatccagcctatggcaaggtgttcaacccaccccggaatatgctgcccggccggttcaccaacttcctggatgtggccgaggcctgtccaacatttctgcacttcgagggcgacgtgccctacgtgaccacaaagaccgactccgatagggtgctggcccagtttgacctgtccctggccgccaagcacatgtctaacacattcctggccggcctggcccagtactatacccagtatagcggcacaatcaatctgcactttatgttcaccggcccaacagacgccaaggcccgctacatgatcgcatatgcacctccaggaatggagccacctaagaccccagaggctgccgcccactgcatccacgcagagtgggataccggcctgaactctaagtttacattcagcatcccctacctgtccgccgccgactacgcctatacagcctccgataccgccgagacaacaaatgtgcagggctgggtgtgcctgtttcagatcacccacggcaaggcagacggcgatgccctggtggtgctggccagcgccggcaaggacttcgagctgaggctgccagtggatgcaaggacccagaccacaagcgccggagagtccgccgaccctgtgaccgccacagtggagaactatggcggcgagacacaggtgcagcggagacagcacacagacgtgagctttatcctggatcggttcgtgaaggtgaccccaaaggaccagatcaatgtgctggatctgatgcagacaccagcacacaccctggtgggcgccctgctgagaaccgccacatactatttcgccgatctggaggtggccgtgaagcacgagggcaacctgacctgggtgcccaatggagcacctgaggccgccctggacaacaccacaaatcccacagcctaccacaaggcacctctgacccgcctggccctgccatatacagcaccacaccgggtgctggcaaccgtgtacaacggcaattccaagtatggcgacggcacagtggccaacgtgagaggcgatctgcaggtgctggcacagaaggcagcaagggccctgccaacctctttcaattacggcgccatcaaggccacccgggtgacagagctgctgtaccggatgaagagagccgagacatattgcccccgccctctgctggcaatccaccctgaccaggcaaggcacaagcagaagatcgtggccccagtgaagcagctgctgaactttgacctgctgaagctggccggcgatgtggagtctaatccaggc

SEQ ID NO: 5 - DNA, 639 bp, synthetic construct
  source 1..639: mol_type = other DNA
tccggagcaccaccaacagatctgcagaagatggtcatgggcaataccaagcccgtggagctgatcctggacggcaagacagtggccatctgctgtgccaccggcgtgtttggcacagcctacctggtgcctcggcacctgttcgccgagaagtatgacaagatcatgctggatggccgcgccatgaccgactccgattaccgggtgtttgagttcgagatcaaggtgaagggccaggacatgctgtctgatgccgccctgatggtgctgcacagaggcaacagggtgcgcgacatcaccaagcactttcgggatacagccagaatgaagaagggcacccctgtggtgggcgtgatcaacaatgccgatgtgggcaggccgatctttagcggcgaggccctgacatacaaggacatcgtggtgtgcatggacggcgataccatgccaggcctgttcgcatacagggcagcaacaaaggcaggatattgtggaggagccgtgctggcaaaggacggagcagataccttcatcgtgggcacacactctgccggcggcaatggcgtgggctattgcagctgtgtgagtaggtctatgctgctgaagatgaaagcccacattgatcctgagccacaccacgag

SEQ ID NO: 6 - DNA, 711 bp, synthetic construct
  source 1..711: mol_type = other DNA
accggtgacatcgaaccccccacacccatctgtcccgaaatttgctcatgcccagctgcagaggtcctgggagcaccgtcggtcttcctcttccctccaaaacccaaggacatcctcatgatctcccggacacccaaggtcacgtgcgtggtggtggacgtgagccaggaggaggctgaagtccagttctcctggtacgtggacggcgtacagttgtacacggcccagacgaggccaatggaggagcagttcaacagcacctaccgcgtggtcagcgtcctgcccatccagcaccaggactggctgaaggggaaggagttcaagtgcaaggtcaacaacaaagacctcctttcccccatcacgaggaccatctccaaggctacagggccgagccgggtgccgcaggtgtacaccctgcccccagcctgggaagagctgtccaagagcaaagtcagcataacctgcctggtcactggcttctacccacctgacatcgatgtcgagtggcagagcaacggacaacaagagccagagggcaattaccgcaccaccccgccccagcaggacgtggatgggacctacttcctgtacagcaagctcgcggtggacaaggtcaggtggcagcgtggagacctattccagtgtgcggtgatgcacgaggctctgcacaaccactacacccagaagtccatctccaagactcagggtaaa

SEQ ID NO: 7 - DNA, 583 bp, synthetic construct
  source 1..583: mol_type = other DNA
gatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgataataccatgattga

SEQ ID NO: 8 - DNA, 22 bp, synthetic construct
  source 1..22: mol_type = other DNA
catttggcaagaattcgccacc

SEQ ID NO: 9 - DNA, 30 bp, synthetic construct
  source 1..30: mol_type = other DNA
gaacaaaaactcatctcagaagaggatctg

SEQ ID NO: 10 - DNA, 24 bp, synthetic construct
  source 1..24: mol_type = other DNA
gactacaaagacgatgacgacaag

(19) United States
(12) Patent Application Publication
(10) Pub. No.: US 2026/0146065 A1
(43) Pub. Date: May 28, 2026

(54) RECOMBINANT PROTEIN COMPRISING PROTEIN DERIVED FROM FOOT-AND-MOUTH DISEASE VIRUS TYPE O CAPSID PROTEIN AND SFC PROTEIN AND USE THEREOF

(71) Applicant: THE INDUSTRY & ACADEMIC COOPERATION IN CHUNGNAM NATIONAL UNIVERSITY (IAC), Daejeon (KR)

(72) Inventor: Hyun-Jin SHIN, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 19/122,750

(22) PCT Filed: Oct. 19, 2023
(86) PCT No.: PCT/KR2023/016269

§ 371 Date: Apr. 21, 2025

(30) Foreign Application Priority Data
Oct. 19, 2022 (KR) 10-2022-0135134

(51) Int. Cl. C07K 14/005; A61K 39/00; A61K 39/135; A61P 37/04

(52) U.S. Cl. CPC C07K 14/005; A61K 39/135; A61P 37/04; A61K 2039/5258; C07K 2319/30; C12N 2770/32122; C12N 2770/32123; C12N 2770/32134

(57) ABSTRACT

The present disclosure relates to a recombinant protein comprising a food and mouth disease virus (FMDV) virus like particle (VLP) and a fragment crystallizable region (Fc) of a swine-derived immunoglobulin linked to the surface of the VLP, and a vaccine composition comprising the recombinant protein. The recombinant protein of the present disclosure may form a self-assembling structure including a virus-like particle using a protein derived from a capsid protein of FMDV, which is an antigenic protein, and a swine Fc protein located on the surface of the virus-like particle, and by using a vaccine composition including the recombinant protein, a specific antibody against FMDV may be effectively produced.

### TECHNICAL FIELD

The present disclosure relates to a recombinant protein including a protein derived from foot and mouth disease virus type O capsid protein and a swine-derived immunoglobulin Fc protein, and a use thereof. The present application claims priority to Korean Patent Application No. 10-2022-0135134, filed on Oct. 19, 2022, with the Korean Intellectual Property Office, the disclosure of which is incorporated in the present specification by reference.

### RELATED ART

Foot and mouth disease virus is a single-stranded, positive-sense RNA virus belonging to the genus Aphthovirus within the family Picornaviridae, and is a virus existing without an envelope and surrounded by a capsid consisting of structural proteins. It is classified into seven serotypes, namely A, O, C, Asia1, SAT1, SAT2, and SAT3, and these major serotypes are further divided into about 200 subtypes, and frequent mutations occur within serotypes or serosubtypes, but cross-immunity does not occur between serotypes, making the prevention and control of the disease development highly challenging.

Currently, inactivated vaccines are widely used worldwide for a foot and mouth disease vaccine. However, the inactivated foot and mouth disease vaccine has the problem that, firstly, it remains dangerous because it is made using the foot and mouth disease pathogen. There is a possibility that vaccination may result in some degree of virus replication in epithelial cells, and that vaccinated livestocks may remain in a carrier state for future actual foot and mouth disease virus infections. Secondly, as an extension of the issue of directly using the foot and mouth disease virus, existing vaccines have the disadvantage of requiring facilities with a high level of quarantine during the manufacturing process. This in itself is problematic because viruses used in laboratories or vaccine manufacturing plants may spread outside and act as a source of infection, and it is difficult to easily decide to manufacture vaccines in countries where foot and mouth disease does not occur. Thirdly, there is the disadvantage of being difficult to verify the efficacy or risk of the vaccine. In other words, animal testing in a shielded facility is always necessary to verify risk, and it is difficult to maintain consistent performance for each manufactured vaccine. Finally, since existing vaccines are made by concentrating the supernatant of cells infected with foot and mouth disease virus, they contain a large amount of non-structural proteins, and it may be difficult to conduct an NSP-antibody test to distinguish between animals actually infected with foot and mouth disease virus and animals that have been vaccinated and have developed immunity. Therefore, the development of a next-generation vaccine against foot and mouth disease virus is necessary, since the vaccine is not made using the entire foot and mouth disease virus, it does not require special facilities, allows for the production of vaccines with uniform performance, and should proceed in a direction that may distinguish vaccinated animals from naturally infected animals.

Foot and mouth disease virus has many variants not only between serotypes but also has many variants within each serotype due to its genetic complexity and diversity, making vaccine production difficult. In addition, since a vaccine for one serotype does not provide protective immunity against another serotype and there are cases where two strains belonging to the same serotype may differ in their genetic sequences by up to 30% of their total genes, it is generally agreed that the foot and mouth disease vaccine should be applied to each serotype. Therefore, it is important to respond quickly to foot and mouth disease vaccines by identifying the virus serotypes prevalent in the area surrounding the outbreak and manufacturing vaccines according to those serotypes. To achieve this, there is a need to improve the current development and production methods for inactivated foot and mouth disease vaccines. Therefore, it is necessary to develop a different type of vaccine that may complement the problems of existing inactivated vaccines and highlight their advantages. In other words, it is necessary to develop a foot and mouth disease vaccine that may be rapidly reproduced according to serotype, does not require special facilities, and allows for the production of a vaccine with uniform performance.

Meanwhile, virus-like particles (VLP) do not contain the genetic material of a virus and therefore do not cause infection, but are composed of viral structural protein molecules that enable the immune system to mount an immune response against a specific pathogen.

Accordingly, the inventors of the present disclosure prepared a virus-like particle, in other words, a recombinant protein, including a capsid protein derived from foot and mouth disease virus, and confirmed the significant immune response induction efficacy of a vaccine composition including the same, thereby completing the present disclosure.

### SUMMARY OF INVENTION

### Technical Problem

One aspect provides a recombinant protein including a protein derived from a food and mouth disease virus (FMDV) capsid and a fragment crystallizable (Fc) region of a swine-derived immunoglobulin.

Another aspect provides a polynucleotide encoding the recombinant protein.

Another aspect provides a vaccine composition for preventing or treating FMDV infection, including the recombinant protein as an active ingredient.

Another aspect provides a method of preventing or treating FMDV infection disease, including administering the vaccine composition to a subject other than a human.

### Solution to Problem

One aspect is to provide a recombinant protein including a protein derived from a food and mouth disease virus (FMDV) capsid and a fragment crystallizable (Fc) region of a swine-derived immunoglobulin.

As used herein, the term “FMDV” refers to a virus belonging to the genus Aphthovirus within the family Picornaviridae, of which seven serotypes are well known: O, A, C, Asia1, SAT1, SAT2, and SAT3. The genome of FMDV consists of about 8,500 bp of positive-sense RNA, and a protein-coding region is largely divided into three parts: P1, P2, and P3. Here, the P1 region functions as a protein that constitutes the viral capsid, such as VP1, VP2, VP3, and VP4. VP1, VP2, and VP3 are the main components exposed to the outside of the capsid, and VP4 plays a role in connecting these capsids. Among FMDV capsid proteins, a G-H loop of VP1 is known to be a main immunogenic region that induces neutralizing antibody production. The P2 and P3 regions include nonstructural proteins (NSPs) that are important for viral maturation and replication. In particular, the P3 region contains 3D^{pol}, a viral RNA genome polymerase essential for viral replication, and 3C protease, an enzyme that cleaves a viral P12A protein, thus could be said to be an essential region for a viral replication process. The viral replication process is characterized by being initiated using a Vpg protein as a primer, and the completion of a RNA genome replication process creates a virus in its complete form through a maturation process such as RNA encapsulation in the procapsid composed of a viral capsid protein pentamer.

The foot and mouth disease virus capsid-derived protein may include a foot and mouth disease virus P12A protein, and the P12A protein refers to a protein including the P1 protein (VP4, VP2, VP3 and VP1) of the foot and mouth disease virus and a 2A region of the P2 protein. 3C protease or 3C protease L127P may cleave the region between VP2 and VP3, between VP3 and VP1, and between VP1 and 2A, as shown in FIG. 1 below.

In an embodiment, the foot and mouth disease virus capsid-derived protein may consist of an amino acid sequence of SEQ ID NO: 1. Any amino acid sequence that exhibits 80% or more, specifically 90% or more, more specifically 95% or more, even more specifically 98% or more, and most specifically 99% or more homology with the sequence and any amino acid sequence that is substantially the same to or exhibits a corresponding efficacy to the protein is included without limitation.

As used herein, the term “Fragment crystallizable region (Fc region)” refers to a fragment crystallizable region present in an antibody or immunoglobulin, which constitutes a tail region of an antibody or immunoglobulin that interacts with a cell surface receptor called an Fc receptor and some proteins of a complement system. The Fc region may be for increasing the efficiency of a vaccine composition including a recombinant protein according to an embodiment, and the Fc region may refer to an Fc region of a swine-derived immunoglobulin.

The Fc region of the swine-derived immunoglobulin may be directly linked or fused to a FMDV capsid-derived protein, or may be linked to a FMDV capsid-derived protein via a linker. The Fc region of the swine-derived immunoglobulin may be linked to the surface of the FMDV capsid-derived protein.

In an embodiment, the Fc region of the swine-derived immunoglobulin may consist of an amino acid sequence of SEQ ID NO: 3. Any amino acid sequence that exhibits 80% or more, specifically 90% or more, more specifically 95% or more, even more specifically 98% or more, and most specifically 99% or more homology with the sequence and any amino acid sequence that is substantially the same to or exhibits a corresponding efficacy to the protein is included without limitation.

In an embodiment, the recombinant protein may further include a 3C protease protein.

The 3C protease cleaves the P12A protein of FMDV to generate three structural proteins (VP0, VP3, and VP1), and VP0 may then be cleaved into VP4 and VP2 by an action of host cell degradative enzymes. These cleaved proteins may then self-assemble to form FMDV-derived capsid particles that does not include a genetic material.

The 3C protease protein may consist of an amino acid sequence of SEQ ID NO: 2. Any amino acid sequence that exhibits 80% or more, specifically 90% or more, more specifically 95% or more, even more specifically 98% or more, and most specifically 99% or more homology with the sequence and any amino acid sequence that is substantially the same to or exhibits a corresponding efficacy to the protein is included without limitation.

As used herein, the term “homology” refers to the degree of similarity of the nucleotide sequence encoding a protein or the amino acid sequence consisting the protein, such that if the homology is sufficiently high, the expression product of the gene and the protein may have the same or similar activity. Additionally, homology may be expressed as a percentage based on a degree to which a given amino acid sequence or nucleotide sequence matches. As used herein, a homologous sequence having the same or similar activity as a given amino acid sequence or nucleotide sequence is expressed as “% homology.” For example, the sequences may be compared using standard software that calculates parameters such as score, identity and similarity, etc., specifically BLAST 2.0, or by hybridization experiments conducted under defined stringent conditions, and the defined appropriate hybridization conditions are within a scope of the relevant technology and may be determined by methods well known to those skilled in the art (for example, J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York).

In an embodiment, the recombinant protein may self-assemble within a host cell to form a virus-like particle. The recombinant protein may be degraded into monomers by 3C protease and then self-assembled within the host cell to form a virus-like particle.

As used herein, the term “virus-like particle (VLP)” may refer to a non-infectious viral subunit that may or may not contain a viral protein. For example, a VLP may refer to a recombinant protein with a virus-like conformation, wherein the VLP self-assembles into a virus-like conformation by binding to structural proteins of the virus, but the genes of the virus may not be incorporated into the VLP during the assembly procedure. VLP with the above property has a form very similar to an actual virus, thus may exhibit high immunogenicity when injected into the body, and since the VLP does not include a viral gene, and the VLP may act as a safe antigen that cannot proliferate in the body.

According to an embodiment, when a vector including a gene encoding the recombinant protein was used to transfect a host cell, it was confirmed that proteins constituting the viral capsid, such as VP1, VP2, VP3, and VP4 constituting the recombinant protein, were expressed, and a recombinant protein in which sFc is bound to VP1 was also expressed. Furthermore, it was confirmed that these constituent proteins subsequently self-assemble within the host cell to form virus-like particles. In addition, according to an embodiment, when the recombinant protein was inoculated into an experimental animal, it was confirmed that it exhibited high neutralizing ability and enhanced the expression level of immune-related factors.

Another aspect is to provide a polynucleotide encoding the recombinant protein of the present disclosure. The same parts as described above also apply to the polynucleotide.

As used herein, the term “polynucleotide” refers to a polymeric substance to which nucleotides are conjugated, such as DNA, which encodes genetic information.

In the present disclosure, a nucleic acid sequence consisting a polynucleotide encoding the protein includes, without limitation, not only a nucleic acid sequence encoding the amino acid set forth in each SEQ ID NO., but also a nucleic acid sequence showing 80% or more, specifically 90% or more, more specifically 95% or more, more specifically 98% or more, and most specifically 99% or more homology to the sequence, as well as a nucleic acid sequence constituting a polynucleotide encoding a protein exhibiting substantially the same or corresponding potency as each of the proteins.

In addition, the polynucleotide encoding the protein may be subject to various modifications in the coding region without changing the amino acid sequence of the protein expressed from the coding region, taking into account the preferred codon in the organism in which the protein is to be expressed due to degeneracy of the codon. Therefore, the polynucleotides may be included without limitation in the nucleic acid sequence encoding the respective proteins. In addition, a probe which may be prepared from the disclosed sequences, for example, a sequence that hybridizes under stringent conditions to a complementary sequence for all or part of the polynucleotide sequence, and encodes a protein that was the same activity as the protein may be included without limitation.

The term “stringent condition” refers to a condition that enable specific hybridization between polynucleotides. These conditions are described in detail in the literature (for example, J. Sambrook et al., same as above). For example, hybridization between genes with high homology, 40% or more, specifically 90% or more, more specifically 95% or more, more specifically 97% or more, and especially specifically 99% or more homology, and conditions in which genes with less homology do not hybridize, or washing conditions for a typical Southern hybridization such as a salt concentration and temperature equivalent to 60° C. 1×SSC, 0.1% SDS, specifically 60° C. 0.1×SSC, 0.1% SDS, more specifically 68° C., 0.1×SSC, 0.1% SDS, which are the conditions for washing once, specifically 2 to 3 times, may be listed.

Hybridization requires that the two polynucleotides have complementary sequences, although mismatch between bases are possible depending on the degree of stringency of the hybridization. The term “complementary” is used to describe the relationship between nucleotide bases that may hybridize with each other. For example, with respect to DNA, adenosine is complementary to thymine and cytosine is complementary to guanine. Therefore, in addition the present application may include substantially similar polynucleotide sequences as well as isolated polynucleotide fragments that are complementary to the entire sequence.

Specifically, a polynucleotide that has homology may be detected using hybridization conditions including a hybridization process at a Tm value of 55° C. and using the conditions described above. In addition, the Tm value may be 60° C., 63° C., or 65° C., but is not limited thereto and may be appropriately adjusted by a person skilled in the art depending on the purpose. The appropriate degree of stringency to hybridize a polynucleotide depends on the length of the polynucleotide and the degree of complementarity, variables that are well known in the art (see Sambrook et al., supra, 9.50-9.51, 11.7-11.8).

The polynucleotide sequence may be provided in the form of a target protein expression cassette.

The expression cassette may enhance the expression efficiency and extracellular secretion efficiency of a target protein in vivo, so that not only may a target protein be stably and efficiently expressed in vivo, but also the target protein is secreted extracellularly, so that it is easy to acquire or has excellent in vivo action efficiency.

As used herein, the term “expression cassette” refers to a unit cassette that may express a target protein for expression/production or secretion, including any combination of one or more genes and sequences regulating their expression, such as various cis-acting transcriptional regulatory elements. The target protein expression cassette of the present disclosure may be used in conjunction with a secretion system. The expression cassette may further include, inside or outside, various factors that may assist in efficient expression of the target protein, such as a promoter, a transcription enhancer, a terminator, an initiator, a non-translated region, a His-tag, a protease recognition site, and components that control the expression of the target protein.

As used herein, the term “target protein” refers to a protein that a person of ordinary skill in the art aims to express, and refers to any protein that may be expressed in a host cell or a target organism by inserting a polynucleotide sequence encoding the protein into the expression cassette, an expression vector including the same, or the expression cassette or expression vector.

In an embodiment, the expression cassette may be operably linked to an AG promoter polynucleotide sequence, a P12A polynucleotide sequence, a sFc polynucleotide sequence, an internal ribosome entry point polynucleotide sequence, and a 3C protease L127P sequence. In an embodiment, the expression cassette may be operably linked to a CMV promoter polynucleotide sequence, a P12A polynucleotide sequence, a sFc polynucleotide sequence, an internal ribosome entry point polynucleotide sequence, and a 3C protease L127P sequence. In an embodiment, the expression cassette may be operably linked to a CMV promoter polynucleotide sequence, a P12A polynucleotide sequence, a sFc polynucleotide sequence, an internal ribosome entry point polynucleotide sequence, and a 3C protease L127P sequence. In an embodiment, the expression cassette may further include a myc polynucleotide sequence and a flag polynucleotide sequence. The myc polynucleotide sequence may consist of SEQ ID NO: 9, and the myc polynucleotide sequence may consist of SEQ ID NO: 10. The approximate structure of an expression cassette according to an embodiment is as shown in FIG. 1B and FIG. 7A.

The polynucleotide may be provided in a form of an expression vector.

As used herein, the term “expression vector” refers to a recombinant vector that may express a target protein when introduced into a suitable host cell, and a genetic construct including essential regulatory elements operably linked to allow expression of a gene insert. The term “operably linked” refers to a functional linkage of a nucleic acid expression control sequence and a nucleic acid sequence encoding a desired protein to perform a general function. The operational linkage with the recombinant vector may be prepared using genetic recombination techniques well known in the art, and site-specific DNA cleavage and linkage may be easily accomplished using enzymes, etc., generally known in the art.

Suitable expression vectors of the present disclosure may include signal sequences for membrane targeting or secretion in addition to expression control elements such as a promoter, initiation codon, termination codon, polyadenylation signal and enhancer. The initiation codon and termination codon are generally considered to be part of the nucleotide sequence encoding an immunogenic target protein, and must be functional in a subject when a genetic construct is administered and must be in frame with the coding sequence. Common promoters may be constitutive or inducible and include, but are not limited to, a lac, tac, T3 and T7 promoters in a prokaryotic cell, and a simian virus 40 (SV40), mouse mammary tumor virus (MMTV) promoters, human immunodeficiency virus (HIV), for example, the long terminal repeat (LTR) promoter of HIV, Moloney virus, cytomegalovirus (CMV), Epstein-Barr virus (EBV), Rous sarcoma virus (RSV) promoters, as well as the β-actin promoter, human hemoglobin, human muscle creatine and human metallothionein-derived promoters, etc., in eukaryotic cells.

Additionally, the expression vector may include a selectable marker for selecting a host cell containing the vector. The selection markers are for selecting cells transformed with the vector, and markers that confer a selectable phenotype, such as drug resistance, nutrient requirement, resistance to cytotoxic agents, or expression of surface proteins, may be used. In an environment treated with a selective agent, only cells expressing the selectable marker survive, thus transformed cells may be selected. Additionally, if the vector is a replicable expression vector, it may include a replication origin, which is a specific nucleic acid sequence at which replication is initiated.

Various forms of vectors, such as plasmids, viruses, and cosmids, etc., may be used as recombinant expression vectors for inserting foreign genes. The type of recombinant vector is not particularly limited as long as it functions to express a desired gene and producing a desired protein in various host cells of prokaryotic cell and eukaryotic cell, however, specifically, a vector that has a promoter that exhibits strong activity and a strong expression ability while being able to mass-produce a foreign protein in a form similar to that in the natural state may be used.

To express the recombinant protein of the present disclosure, various combinations of hosts and vectors may be used. Expression vectors suitable for eukaryotic hosts may include, but are not limited to, expression control sequences, etc., derived from SV40, bovine papillomavirus, adenovirus, adenoassociated virus, cytomegalovirus, and retrovirus. Expression vectors that may be used in bacterial hosts include, but are not limited to, bacterial plasmids obtained from Escherichia coli including pcDNA3.1, pET, pRSET, pBluescript, pGEX2T, pUC vectors, col E1, pCR1, pBR322, pMB9 or derivatives thereof, etc., plasmids having a broader host range such as RP4, phage DNA such as phage lambda derivatives such as Agt10, Agt11 or NM989, and other DNA phages such as M13 and filamentous single-stranded DNA phages, etc. For yeast cells, 2° C. plasmid or its derivatives, etc., may be used, and for insect cells, pVL941, etc. may be used.

Another aspect is to provide a vaccine composition for preventing or treating FMDV infection, including a recombinant protein according to an embodiment as an active ingredient. The same parts as described above also apply to the composition.

As used herein, the term “vaccine” refers to a pharmaceutical composition containing at least one immunologically active component that induces an immunological response in an animal. The immunologically active component of the vaccine may contain suitable elements of live or dead virus (subunit vaccines), whereby these elements are prepared by destruction of whole virus or a growth culture thereof, followed by a purification step to acquire a desired construct(s), or by a synthetic process induced by suitable manipulation of a suitable system, such as but not limited to bacteria, insects, mammals or other species, followed by isolation and purification, or by induction of said synthetic process in an animal in need of the vaccine by direct incorporation of the genetic material using a suitable pharmaceutical composition (polynucleotide vaccination). The vaccine may include one or more of the elements described above, either individually or at the same time.

As used herein, the term “prevention” refers to any act of inhibiting or delaying infection with FMDV and the onset of disease caused by said infection by administration of an FMDV vaccine composition.

As used herein, the term “treatment” refers to any action that improves or benefits the symptoms of a disease already caused by infection with FMDV due to administration of an FMDV vaccine composition.

The vaccine composition may additionally include a pharmaceutically acceptable excipient, diluent, or carrier. The term “pharmaceutically acceptable excipient, diluent or carrier” may refer to an excipient, diluent or carrier that does not irritate living organisms and does not inhibit the biological activity and properties of the injected compound. Here, “pharmaceutically accepted” means that it does not inhibit the activity of the active ingredient and does not have any toxicity beyond what the subject of application (prescription) may adapt to.

Suitable carrier for a vaccine is known to those skilled in the art and include, but are not limited to, protein, sugar, etc. The carrier may be an aqueous solution or non-aqueous solution, suspension or emulsion. Structured or unstructured organic or inorganic polymer, etc. may be used as an immune adjuvant to increase immunogenicity. Immune adjuvants are generally known to play a role in promoting immune responses through chemical and physical binding to an antigen. As an immune adjuvant, an amorphous aluminum gel, oil emulsion, double oil emulsion, immunosol, etc. may be used. In addition, various plant-derived saponin, levamisole, CpG dinucleotide, RNA, DNA, LPS, and various types of cytokines, etc. may be used to promote the immune response. The immune composition as described above may be used as a composition for inducing an optimal immune response by combining various adjuvants and additives to promote immune response. In addition, compositions that may be added to the vaccine include a stabilizer, inactivator, antibiotic, preservative, etc. Depending on the route of administration of the vaccine, the vaccine antigen may also be mixed with distilled water, buffer solution, etc.

The vaccine compositions may be formulated and used in the form of an oral dosage form such as a pill, granule, tablet, capsule, suspension, emulsion, syrup, aerosol, etc., topical preparation, suppository, unit dosage ampoule, or injectable formulation in the form of multiple dosages each according to typical methods. The vaccine composition may be formulated with the addition of a diluent or excipient such as commonly used filler, extender, binder, lubricant, disintegrating agent, or surfactant, etc.

If the vaccine composition is prepared as a parenteral formulation, the vaccine composition may be formulated in the form of an injectable formulation, transdermal administration, nasal inhalant, and suppository along with a suitable carrier according to methods known in the art. When formulated as an injectable formulation, suitable carriers may include sterile water, ethanol, polyols such as glycerol or propylene glycol, or mixtures thereof, desirably Ringer's solution, phosphate buffered saline (PBS) containing triethanolamine, sterile water for injection, and isotonic solutions such as 5% dextrose, etc. may be used. When formulated as a transdermal agent, the transdermal agent may be formulated in the form of an ointment, cream, lotion, gel, topical solution, paste, liniment, and aerosol, etc. For a nasal inhalant, the nasal inhalant may be formulated in the form of an aerosol spray using a suitable propellant such as dichlorofluoromethane, trichlorofluoromethane, dichlorotetrafluoroethane, carbon dioxide, etc., when formulated as a suppository, the base used may be witepsol, tween 61, polyethylene glycol, cacao butter, laurin butter, polyoxyethylene sorbitan fatty acid esters, polyoxyethylene stearate, sorbitan fatty acid esters, etc.

Another aspect provides a method of preventing or treating FMDV infection disease, including administering the vaccine composition to a subject other than a human. The same aspects as described above apply equally to the method.

As used herein, the term “subject” refers to a living organism that may be infected with FMDV and may develop a disease due to the infected FMDV, preferably, but not particularly limited to, a mammal. The mammal may include a cow, a sheep, a pig, a goat, a camel, an antelope, etc., and may be specifically a pig.

As used herein, the term “administration” refers to introducing a certain substance into a subject by an appropriate method, and the administration route of the vaccine composition of the present disclosure may be administered through any general route as long as the substance may reach the target tissue. Routes of administration may include, but not limited to, intraperitoneal administration, intravenous administration, intramuscular administration, subcutaneous administration, intradermal administration, oral administration, topical administration, intranasal administration, intrapulmonary administration, and rectal administration. However, since proteins are digested when administered orally, it is desirable for oral compositions to be coated with the active agent or formulated to protect them from decomposition in the stomach. In addition, a pharmaceutical composition may be administered by any device that may transport the active substance to target cells.

The administration route of the vaccine composition may be via any general route as long as the vaccine composition may reach the target tissue, and more specifically, the vaccine composition may be selected from the group consisting of compositions for intramuscular administration, subcutaneous administration, intraperitoneal administration, intravenous administration, oral administration, dermal administration, ocular administration, and intracerebral administration.

The vaccine composition may be administered in a pharmaceutically effective amount, wherein the term “pharmaceutically effective amount” refers to an amount sufficient to treat or prevent a disease with a reasonable benefit/risk ratio applicable to medical treatment or prevention, and an effective dose level may be determined according to factors including severity of the disease, activity of a drug, age, weight, health, and sex of a patient, sensitivity of the patient to a drug, time of administration, route of administration, and elimination rate of the composition of the disclosure used, duration of treatment, the drugs used in combination or concurrently with the composition of the disclosure, and other factors well known in the medical field. The vaccine composition may be administered alone or in combination with a component known to exhibit a preventive or therapeutic effect against a known FMDV infectious disease. Taking all of the factors into account, it is important to administer the minimum amount that may achieve the maximum effect without side effects.

The dosage of the vaccine composition may be determined by a person skilled in the art considering the purpose of use, the degree of addiction of the disease, the patient's age, weight, gender, pre-existing condition, or the type of substance used as an active ingredient, etc. For example, the vaccine compositions of the present disclosure may be administered at a dose of from about 0.1 ng to about 1,000 mg/kg per adult, desirably from 1 ng to about 100 mg/kg, and the frequency of administration of the compositions of the present disclosure is not specifically limited, but may be administered once daily or administered multiple times in divided doses. The dosage or frequency of administration is not intended to limit the scope of the present disclosure in any way.

The vaccine composition may be administered as an individual therapeutic agent or administered in combination with other therapeutic agents and may be administered sequentially or simultaneously with typical therapeutic agents. And may be administered in single or multiple doses. Taking all of the factors into consideration, it is important to administer an amount that may achieve maximum effect with the minimum amount without side effects, and may be readily determined by those skilled in the art.

### Effects of Invention

A recombinant protein of the present disclosure may form a self-assembling structure including a virus analogue using a protein derived from a capsid protein of FMDV, which is an antigenic protein, and an immune-enhancing substance located on a surface of an analogue, and by using a vaccine composition including the recombinant protein, specific antibodies against FMDV may be effectively produced

### BRIEF DESCRIPTION OF DRAWINGS

FIG. 1 illustrates components of a recombinant protein according to an embodiment.

FIG. 2 schematically illustrates a structure of an expression cassette according to an embodiment.

FIG. 3 schematically illustrates a structure of an FDMV VLP-sFc structure according to an embodiment.

FIG. 4 and FIG. 5 show the results of confirming an expression of a recombinant protein and its components according to an embodiment through western blotting.

FIG. 6 and FIG. 7 show the results of confirming an expression of a recombinant protein and its components that have undergone purification and concentration according to an embodiment through western blotting.

FIG. 8 shows the results of comparing the sizes of two VLPs through dynamic light scattering.

FIG. 9 and FIG. 10 show the results of comparing the diameters of two VLPs through atomic force microscopy (AFM) images.

FIG. 11 and FIG. 12 show the results of confirming FMDV VLP and FMDV VLP-sFc according to an embodiment using a transmission electron microscope (TEM).

FIG. 13 schematically illustrates a structure of a plasmid for expressing FMDV VLP and FMDV VLP-sFc recombinant proteins according to an embodiment.

FIG. 14 shows the results of confirming an expression of a recombinant protein and its components acquired from a recombinant vector according to an embodiment through western blotting.

FIG. 15 is a diagram schematically illustrating a schedule for evaluating the efficacy of a vaccine composition according to an embodiment.

FIG. 16 to FIG. 20 show the results of measuring the immunogenicity of a vaccine composition according to an embodiment.

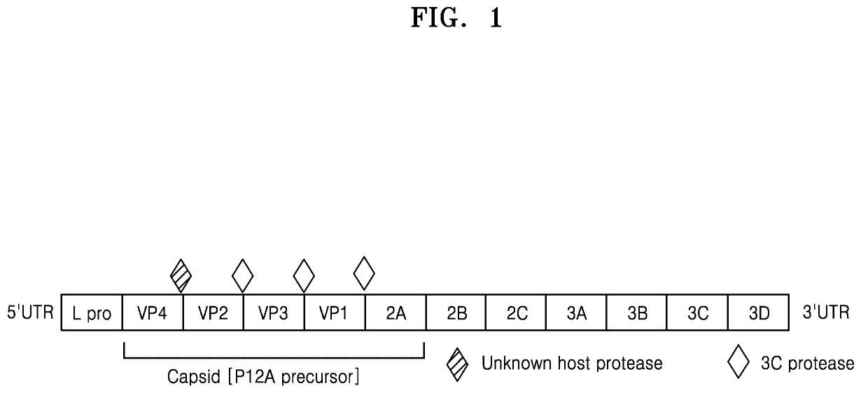

### DESCRIPTION OF EMBODIMENTS

The present disclosure will be explained in more detail in the following examples. However, these examples are for illustrative purposes only and the scope of the present disclosure is not limited to these examples.

### Example 1: Construction of Recombinant Expression Vectors for Preparing FMDV VLP and FMDV VLP-sFc Recombinant Proteins

In the present example, to prepare FMDV VLP and FMDV VLP-sFc recombinant proteins, a gene for a P12A protein, which is a capsid-derived protein of FMDV, a gene for a 3C protease (3C protease L127P) protein based on a O1-Manisa strain (GenBank: AY593823.1), a gene for a swine-derived immunoglobulin Fc region (swine Fc, sFc) protein, and an encephalomyocarditis virus (ECMV) internal ribosomal entry site (IRES) gene were synthesized through the Genescript gene synthesis service. A human codon-optimized polynucleotide sequence for FDMV P12A precursor, a human codon-optimized polynucleotide sequence for FMDV 3C protease L127P, a sFc polynucleotide sequence, and an IRES polynucleotide sequence are respectively listed in SEQ ID NO: 4 to 7 in Table 1 below.

The amino acid sequence of the P12A protein, a capsid-derived protein of FMDV, the amino acid sequence of the 3C protease, and the amino acid sequence of the swine-derived immunoglobulin Fc region protein are respectively listed in SEQ ID NO: 1 to 3 in Table 2 below.

TABLE 1

| Name | Sequence Information | SEQ ID NO. |
|---|---|---|
| FMDV | GGAGCAGGACAGAGCTCCCCTGCAACCGGCTCT | 4 |
| P12A | CAGAACCAGAGCGGCAATACAGGCAGCATCATC | |
| precursor | AACAATTACTATATGCAGCAGTATCAGAACTCCA | |
| human | TGGACACCCAGCTGGGCGATAACGCCACAAGCG | |
| codon | GCGGCTCCAATGAGGGCTCCACAGATACCACAT | |
| optimized | CTACCCACACCACAAATACACAGAACAATGACT | |
| | GGTTTAGCAAGCTGGCCTCTAGCGCCTTTTCCGG | |
| | CCTGTTCGGCGCCCTGCTGGCAGATAAGAAGACC | |
| | GAGGAGACAACCCTGCTGGAGGACAGAATCCTG | |
| | ACCACACGCAACGGCCACACCACATCCACCACA | |
| | CAGTCCTCTGTGGGCGTGACCTACGGCTATGCAA | |
| | CAGCAGAGGACTTCGTGAGCGGACCAAATACCT | |
| | CCGGCCTGGAGACAAGGGTGGCACAGGCAGAGC | |
| | GGTTCTTCAAGACCCACCTGTTTGACTGGGTGAC | |
| | AAGCGATCCCTTCGGCCGGTGCCACCTGCTGGAG | |
| | CTGCCTACCGACCACAAGGGCGTGTACGGCTCCC | |
| | TGACAGATTCTTACGCCTATATGAGAAACGGATG | |
| | GGACGTGGAGGTGACCGCAGTGGGAAACCAGTT | |
| | TAATGGAGGATGCCTGCTGGTGGCAATGGTGCCT | |
| | GAGCTGTGCTCCATCCAGAAGCGCGAGCTGTATC | |
| | AGCTGACCCTGTTTCCCCACCAGTTCATCAACCC | |
| | TCGGACCAATATGACAGCCCACATCACAGTGCCT | |
| | TTCGTGGGCGTGAACAGATACGACCAGTATAAG | |
| | GTGCACAAGCCATGGACCCTGGTGGTCATGGTG | |
| | GTGGCCCCTCTGACAGTGAATAGCGAGGGCGCC | |
| | CCACAGATCAAGGTGTACGCCAACATCGCCCCA | |
| | ACCAATGTGCACGTGGCCGGCGAGTTTCCTAGCA | |
| | AGGAGGGCATCTTCCCAGTGGCCTGCTCCGATGG | |
| | ATACGGAGGCCTGGTGACCACAGACCCTAAGAC | |
| | CGCCGATCCAGCCTATGGCAAGGTGTTCAACCCA | |
| | CCCCGGAATATGCTGCCCGGCCGGTTCACCAACT | |
| | TCCTGGATGTGGCCGAGGCCTGTCCAACATTTCT | |
| | GCACTTCGAGGGCGACGTGCCCTACGTGACCAC | |
| | AAAGACCGACTCCGATAGGGTGCTGGCCCAGTTT | |
| | GACCTGTCCCTGGCCGCCAAGCACATGTCTAACA | |
| | CATTCCTGGCCGGCCTGGCCCAGTACTATACCCA | |
| | GTATAGCGGCACAATCAATCTGCACTTTATGTTC | |
| | ACCGGCCCAACAGACGCCAAGGCCCGCTACATG | |
| | ATCGCATATGCACCTCCAGGAATGGAGCCACCTA | |
| | AGACCCCAGAGGCTGCCGCCCACTGCATCCACG | |
| | CAGAGTGGGATACCGGCCTGAACTCTAAGTTTAC | |
| | ATTCAGCATCCCCTACCTGTCCGCCGCCGACTAC | |
| | GCCTATACAGCCTCCGATACCGCCGAGACAACA | |
| | AATGTGCAGGGCTGGGTGTGCCTGTTTCAGATCA | |
| | CCCACGGCAAGGCAGACGGCGATGCCCTGGTGG | |
| | TGCTGGCCAGCGCCGGCAAGGACTTCGAGCTGA | |
| | GGCTGCCAGTGGATGCAAGGACCCAGACCACAA | |
| | GCGCCGGAGAGTCCGCCGACCCTGTGACCGCCA | |
| | CAGTGGAGAACTATGGCGGCGAGACACAGGTGC | |
| | AGCGGAGACAGCACACAGACGTGAGCTTTATCC | |
| | TGGATCGGTTCGTGAAGGTGACCCCAAAGGACC | |
| | AGATCAATGTGCTGGATCTGATGCAGACACCAG | |
| | CACACACCCTGGTGGGCGCCCTGCTGAGAACCG | |
| | CCACATACTATTTCGCCGATCTGGAGGTGGCCGT | |
| | GAAGCACGAGGGCAACCTGACCTGGGTGCCCAA | |
| | TGGAGCACCTGAGGCCGCCCTGGACAACACCAC | |
| | AAATCCCACAGCCTACCACAAGGCACCTCTGACC | |
| | CGCCTGGCCCTGCCATATACAGCACCACACCGGG | |
| | TGCTGGCAACCGTGTACAACGGCAATTCCAAGTA | |
| | TGGCGACGGCACAGTGGCCAACGTGAGAGGCGA | |
| | TCTGCAGGTGCTGGCACAGAAGGCAGCAAGGGC | |
| | CCTGCCAACCTCTTTCAATTACGGCGCCATCAAG | |
| | GCCACCCGGGTGACAGAGCTGCTGTACCGGATG | |
| | AAGAGAGCCGAGACATATTGCCCCCGCCCTCTGC | |
| | TGGCAATCCACCCTGACCAGGCAAGGCACAAGC | |
| | AGAAGATCGTGGCCCCAGTGAAGCAGCTGCTGA | |
| | ACTTTGACCTGCTGAAGCTGGCCGGCGATGTGGA | |
| | GTCTAATCCAGGC | |
| FMDV 3C | TCCGGAGCACCACCAACAGATCTGCAGAAGATG | 5 |
| protease | GTCATGGGCAATACCAAGCCCGTGGAGCTGATC | |
| L127P | CTGGACGGCAAGACAGTGGCCATCTGCTGTGCC | |
| human | ACCGGCGTGTTTGGCACAGCCTACCTGGTGCCTC | |
| codon | GGCACCTGTTCGCCGAGAAGTATGACAAGATCA | |
| optimized | TGCTGGATGGCCGCGCCATGACCGACTCCGATTA | |
| | CCGGGTGTTTGAGTTCGAGATCAAGGTGAAGGG | |
| | CCAGGACATGCTGTCTGATGCCGCCCTGATGGTG | |
| | CTGCACAGAGGCAACAGGGTGCGCGACATCACC | |
| | AAGCACTTTCGGGATACAGCCAGAATGAAGAAG | |
| | GGCACCCCTGTGGTGGGCGTGATCAACAATGCC | |
| | GATGTGGGCAGGCCGATCTTTAGCGGCGAGGCC | |
| | CTGACATACAAGGACATCGTGGTGTGCATGGAC | |
| | GGCGATACCATGCCAGGCCTGTTCGCATACAGG | |
| | GCAGCAACAAAGGCAGGATATTGTGGAGGAGCC | |
| | GTGCTGGCAAAGGACGGAGCAGATACCTTCATC | |
| | GTGGGCACACACTCTGCCGGCGGCAATGGCGTG | |
| | GGCTATTGCAGCTGTGTGAGTAGGTCTATGCTGC | |
| | TGAAGATGAAAGCCCACATTGATCCTGAGCCAC | |
| | ACCACGAG | |
| Swine Fc | ACCGGTGACATCGAACCCCCCACACCCATCTGTC | 6 |
| region | CCGAAATTTGCTCATGCCCAGCTGCAGAGGTCCT | |
| sequence | GGGAGCACCGTCGGTCTTCCTCTTCCCTCCAAAA | |
| | CCCAAGGACATCCTCATGATCTCCCGGACACCCA | |
| | AGGTCACGTGCGTGGTGGTGGACGTGAGCCAGG | |
| | AGGAGGCTGAAGTCCAGTTCTCCTGGTACGTGGA | |
| | CGGCGTACAGTTGTACACGGCCCAGACGAGGCC | |
| | AATGGAGGAGCAGTTCAACAGCACCTACCGCGT | |
| | GGTCAGCGTCCTGCCCATCCAGCACCAGGACTGG | |
| | CTGAAGGGGAAGGAGTTCAAGTGCAAGGTCAAC | |
| | AACAAAGACCTCCTTTCCCCCATCACGAGGACCA | |
| | TCTCCAAGGCTACAGGGCCGAGCCGGGTGCCGC | |
| | AGGTGTACACCCTGCCCCCAGCCTGGGAAGAGC | |
| | TGTCCAAGAGCAAAGTCAGCATAACCTGCCTGGT | |
| | CACTGGCTTCTACCCACCTGACATCGATGTCGAG | |
| | TGGCAGAGCAACGGACAACAAGAGCCAGAGGGC | |
| | AATTACCGCACCACCCCGCCCCAGCAGGACGTG | |
| | GATGGGACCTACTTCCTGTACAGCAAGCTCGCGG | |
| | TGGACAAGGTCAGGTGGCAGCGTGGAGACCTAT | |
| | TCCAGTGTGCGGTGATGCACGAGGCTCTGCACAA | |
| | CCACTACACCCAGAAGTCCATCTCCAAGACTCAG | |
| | GGTAAA | |
| IRES | GATCAATTCCGCCCCCCCCCCCTAACGTTACTGG | 7 |
| | CCGAAGCCGCTTGGAATAAGGCCGGTGTGCGTTT | |
| | GTCTATATGTTATTTTCCACCATATTGCCGTCTTT | |
| | TGGCAATGTGAGGGCCCGGAAACCTGGCCCTGT | |
| | CTTCTTGACGAGCATTCCTAGGGGTCTTTCCCCTC | |
| | TCGCCAAAGGAATGCAAGGTCTGTTGAATGTCGT | |
| | GAAGGAAGCAGTTCCTCTGGAAGCTTCTTGAAG | |
| | ACAAACAACGTCTGTAGCGACCCTTTGCAGGCA | |
| | GCGGAACCCCCCACCTGGCGACAGGTGCCTCTGC | |
| | GGCCAAAAGCCACGTGTATAAGATACACCTGCA | |
| | AAGGCGGCACAACCCCAGTGCCACGTTGTGAGT | |
| | TGGATAGTTGTGGAAAGAGTCAAATGGCTCTCCT | |
| | CAAGCGTATTCAACAAGGGGCTGAAGGATGCCC | |
| | AGAAGGTACCCCATTGTATGGGATCTGATCTGGG | |
| | GCCTCGGTGCACATGCTTTACGTGTGTTTAGTCG | |
| | AGGTTAAAAAACGTCTAGGCCCCCCGAACCACG | |
| | GGGACGTGGTTTTCCTTTGAAAAACACGATAATA | |
| | CCATGATTGA | |

TABLE 2

| Name | Sequence Information | SEQ ID NO |
|---|---|---|
| FMDV | GAGQSSPATGSQNQSGNTGSIINNYYMQQYQNSM | 1 |
| capsid | DTQLGDNATSGGSNEGSTDTTSTHTTNTQNNDWFS | |
| P12A | KLASSAFSGLFGALLADKKTEETTLLEDRILTTRNG | |
| | HTTSTTQSSVGVTYGYATAEDFVSGPNTSGLETRV | |
| | AQAERFFKTHLFDWVTSDPFGRCHLLELPTDHKGV | |
| | YGSLTDSYAYMRNGWDVEVTAVGNQFNGGCLLV | |
| | AMVPELCSIQKRELYQLTLFPHQFINPRTNMTAHIT | |
| | VPFVGVNRYDQYKVHKPWTLVVMVVAPLTVNSE | |
| | GAPQIKVYANIAPTNVHVAGEFPSKEGIFPVACSDG | |
| | YGGLVTTDPKTADPAYGKVFNPPRNMLPGRFTNFL | |
| | DVAEACPTFLHFEGDVPYVTTKTDSDRVLAQFDLS | |
| | LAAKHMSNTFLAGLAQYYTQYSGTINLHFMFTGPT | |
| | DAKARYMIAYAPPGMEPPKTPEAAAHCIHAEWDT | |
| | GLNSKFTFSIPYLSAADYAYTASDTAETTNVQGWV | |
| | CLFQITHGKADGDALVVLASAGKDFELRLPVDART | |
| | QTTSAGESADPVTATVENYGGETQVQRRQHTDVS | |
| | FILDRFVKVTPKDQINVLDLMQTPAHTLVGALLRT | |
| | ATYYFADLEVAVKHEGNLTWVPNGAPEAALDNTT | |
| | NPTAYHKAPLTRLALPYTAPHRVLATVYNGNSKY | |
| | GDGTVANVRGDLQVLAQKAARALPTSFNYGAIKA | |
| | TRVTELLYRMKRAETYCPRPLLAIHPDQARHKQKI | |
| | VAPVKQLLNFDLLKLAGDVESNPG | |
| 3C protease | SGAPPTDLQKMVMGNTKPVELILDGKTVAICCATG | |
| L127P | VFGTAYLVPRHLFAEKYDKIMLDGRAMTDSDYRV | |
| | FEFEIKVKGQDMLSDAALMVLHRGNRVRDITKHFR | 2 |
| | DTARMKKGTPVVGVINNADVGRPIFSGEALTYKDI | |
| | VVCMDGDTMPGLFAYRAATKAGYCGGAVLAKDG | |
| | ADTFIVGTHSAGGNGVGYCSCVSRSMLLKMKAHI | |
| | DPEPHHE | |
| swine Fc | DIEPPTPICPEICSCPAAEVLGAPSVFLFPPKPKDILMI | 3 |
| | SRTPKVTCVVVDVSQEEAEVQFSWYVDGVQLYTA | |
| | QTRPMEEQFNSTYRVVSVLPIQHQDWLKGKEFKC | |
| | KVNNKDLLSPITRTISKATGPSRVPQVYTLPPAWEE | |
| | LSKSKVSITCLVTGFYPPDIDVEWQSNGQQEPEGNY | |
| | RTTPPQQDVDGTYFLYSKLAVDKVRWQRGDLFQC | |
| | AVMHEALHNHYTQKSISKTQGKEQKLISEEDL | |

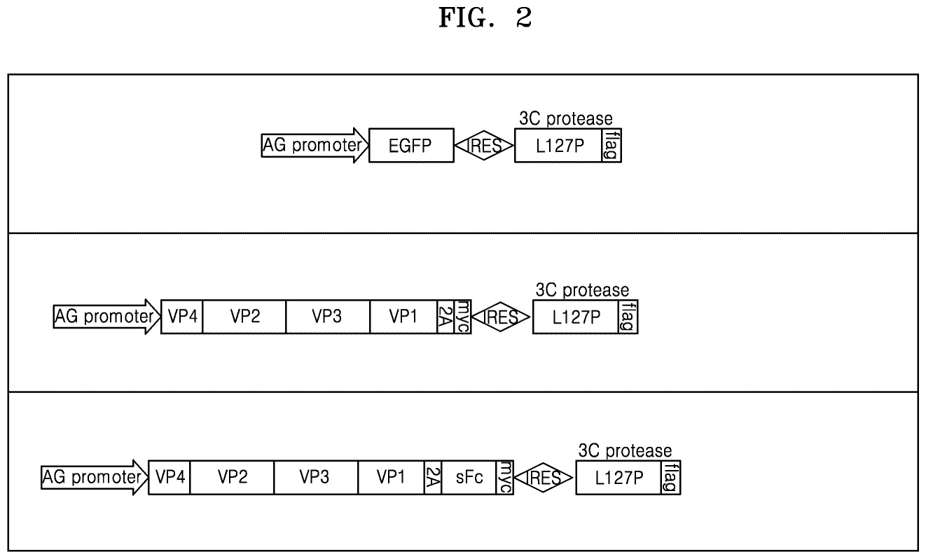

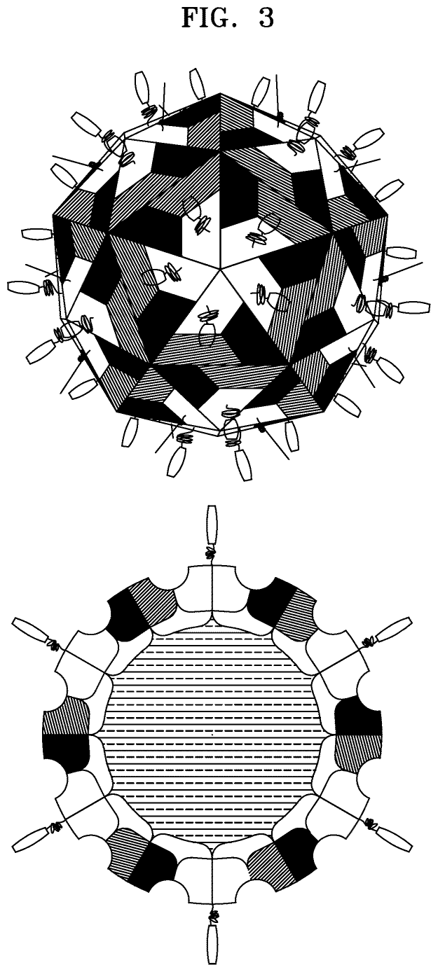

Thereafter, an expression cassette including the synthesized polynucleotide sequence was cloned into a backbone vector pCAGGS, and a vector including genes shown in Table 3 below was acquired. The structure of the expression cassette used here is shown in FIG. 2.

TABLE 3

| | Gene Information Inserted |
|---|---|
| Vector Name | Into the Vector |
| pCAGGS EGFP IRES 3C protease | EGFP, IRES, 3C protease |
| pCAGGS P12A IRES 3C protease | P12A, IRES, 3C protease |
| pCAGGS P12A-sFc IRES 3C protease | P12, sFc, IRES and 3C protease |

### Example 2: Preparation and Verification of FMDV VLP and FMDV VLP-Fc Recombinant Protein

In the present example, a virus-like particle was prepared using the recombinant expression vector of Example 1.

### 2-1. Purification of Recombinant Protein

Specifically, HEK293A (Thermo Fisher Scientific, R70507) cells were cultured in DMEM (Dulbecco's Modified Eagle's Medium) supplemented with 10% fetal bovine serum (Gibco, 16000044) and 1× Antibiotic/Antimycotic (Gibco, 15240062) and stored at 5% CO₂ and 37° C. HEK293A cells cultured in this manner were transfected with each of a pCAGGS P12A IRES 3C protease recombinant expression vector and a pCAGGS P12A-sFc IRES 3C protease recombinant expression vector, using polyethylenimine (PEI; Polysciences, 239966-2) at a DNA:PEI=3:1 ratio for 72 hours. A total of 8 μg of DNA was used per 100 mm dish. As a control group, a group transformed with a pCAGGS EGFP IRES 3C protease recombinant expression vector using the above method was used.

The transfected cells were then pelleted at 1,500 g for 10 minutes at 4° C., resuspended in phosphate buffer solution (40 mM sodium phosphate, 100 mM NaCl pH 7.6) and lysed with 0.5% NP-40 (Sigma Aldrich, NP40S) on ice for 30 minutes. The lysate was purified at 12,000 g for 20 minutes at 4° C., and then western blotting was conducted as follows.

The experimental method of western blotting is as follows. The transformed cells were washed with cold PBS and lysed in RIPA buffer (Thermo Fisher, 89900) containing protease inhibitor cocktail (Sigma Aldrich, P8215) at 4° C. for 30 minutes, and then a supernatant was collected. Protein concentration was measured using a BCA protein assay kit (Thermo Fisher, 23227). Simply put, equal amounts of proteins were separated on a polyacrylamide-tricine gel (10% polyacrylamide). After SDS-PAGE, the gel was transferred to a 0.45 μm polyvinylidene fluoride membrane (Millipore, IPVH00010) and blocked with 5% BSA in TBST (TBS including 0.1% Tween 20) for 1 hour at room temperature (RT). The membrane was incubated with a primary antibody overnight at 4° C. After washing the membrane with TBST, the membrane was incubated with HRP-tagged anti-rabbit IgG (1:10000 dilution) for 2 hour at RT. Image observations were performed using an ATTO Luminograph (Japan) with ECL solution (SuperSignal West Femto Maximum Sensitivity Substrate, 34095). Antibodies used in this experiment were as follows: anti-myc antibody (Cusabio, CSB-PA000085), anti-Flag antibody (Cusabio, CSB-MA000156), anti-swine IgG Fc (Abcam, ab112637), horseradish peroxidase (HRP)-conjugated anti-swine IgG Fc (Abcam, ab112748), HRP-conjugated anti-mouse IgG (Cusabio, CSB-PA573747), HRP-conjugated anti-rabbit IgG (Cusabio, CSB-PA564648).

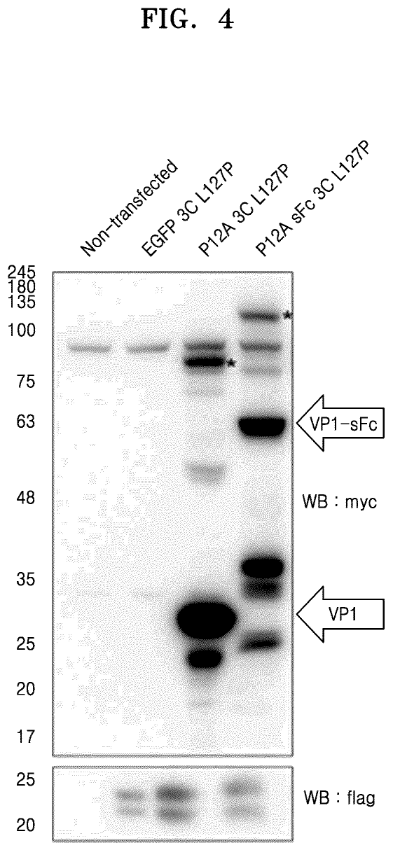

As a result, as shown in FIG. 4, an 85 kDa (uncleaved P12A precursor) band and a 28 kDa (cleaved VP1) band were observed in the pCAGGS P12A IRES 3C protease group (lane 3). Additionally, a 120 kDa (uncleaved P12A-sFc) and a 63 kDa (cleaved VP1-sFc) band were observed in a pCAGGS P12A-sFc IRES 3C protease group (lane 4). These results indicate that, in cells transfected with the recombinant expression vector, both uncleaved P12A-sFc and cleaved recombinant protein in a form of VP1-sFc are expressed.

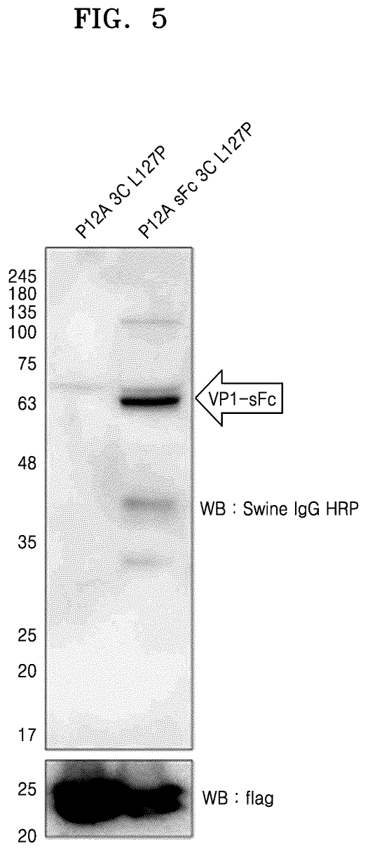

In addition, an expression of sFc was confirmed using anti-swine IgG antibody (FIG. 5). As shown in FIG. 5, it was confirmed that a distinct band of 63 kDa was observed only in the pCAGGS P12A-sFc IRES 3C protease group (lane 2).

### 2-2. Purification and Concentration of Recombinant Protein

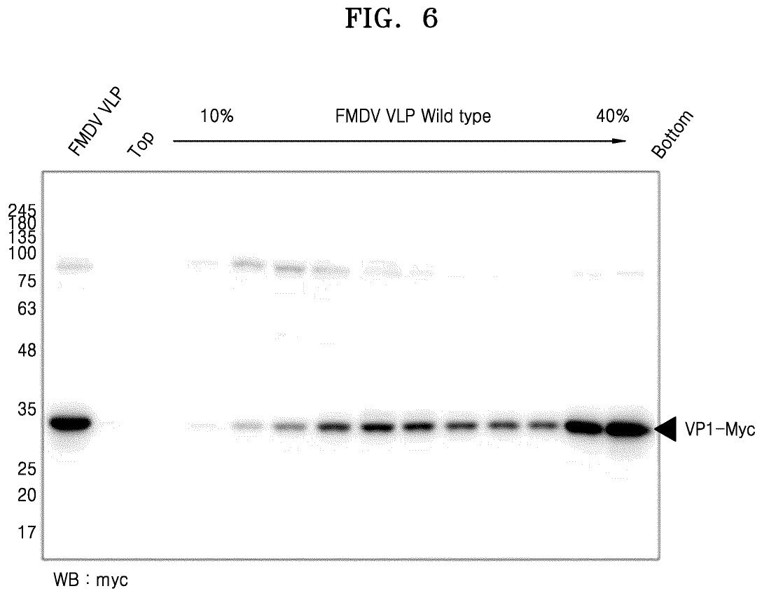

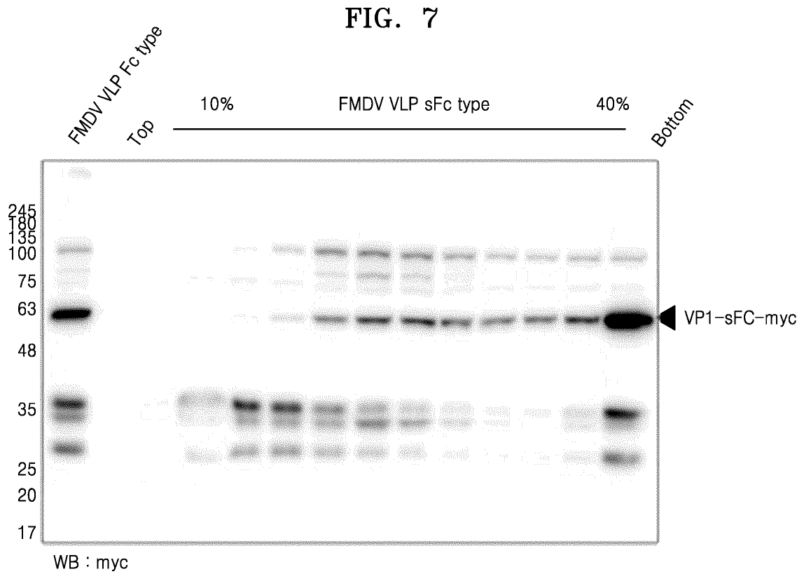

In addition, a supernatant acquired from the above Example 2-1 was loaded onto a 10-40% sucrose medium for ultracentrifugation, and after ultracentrifugation at 250,000 g for 18 hours at 10° C., the gradient was fractionated, and western blotting was conducted on the gradient. A recombinant protein was acquired by desalting using a spin column (Thermo Fisher Scientific, 89882) to remove sucrose and concentrating using an Amicon® Ultra 100 kDa centrifuge filter (Merck Millipore, UFC900396). Western blotting was conducted on the recombinant protein that had undergone a purification and concentration process in this manner, and the results are shown in FIG. 6 and FIG. 7.

As a result, it was confirmed that the cleaved VP1-sFc structure was detected, as shown in FIG. 7.

Through this, it was confirmed that a FMDV VLP-sFc recombinant protein according to an embodiment may be cleaved by protease 3C to generate a VP1-sFc structural unit. These results indicate that the recombinant proteins according to an embodiment may each be cleaved into structural proteins and then formed into capsid subunits by self-assembly, successfully generating virus-like particles.

### Example 3: Confirmation of sFc-Tagged Virus-Like Particles Using Dynamic Light Scattering (DLS) Technique

The diameter of a protein in a sample was measured using dynamic light scattering (DLS).

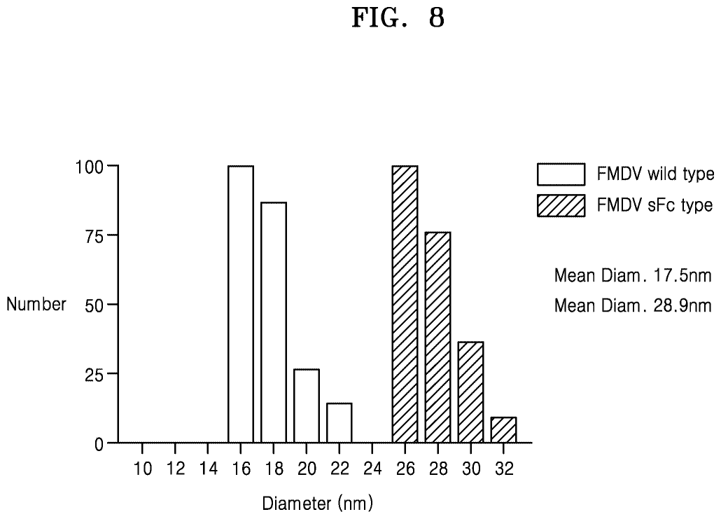

Specifically, as a result of comparing the diameters of virus-like particles generated from a FMDV VLP and FMDV VLP-sFc recombinant protein prepared in Example 2-2, it was confirmed that FMDV VLP produced particles with a diameter of 16 to 22 nm, and FMDV VLP-sFc produced particles with a diameter of 26 to 31 nm. These results indicate that sFc was successfully attached to a VLP surface (FIG. 8).

### Example 4: Confirmation of sFc-Tagged Virus-Like Particles Using Atomic Force Microscopy (AFM) Technique

Atomic Force Microscopy (AFM) is a technique for measuring the exact size of proteins, in other words, height and diameter. This is a specific technique that may be used to measure the diameter and radius of the protein size in a sample. The FMDV VLP and FMDV VLP-sFc recombinant proteins were compared using AFM software (XE-100; Park Systems Co., Suwon, Korea).

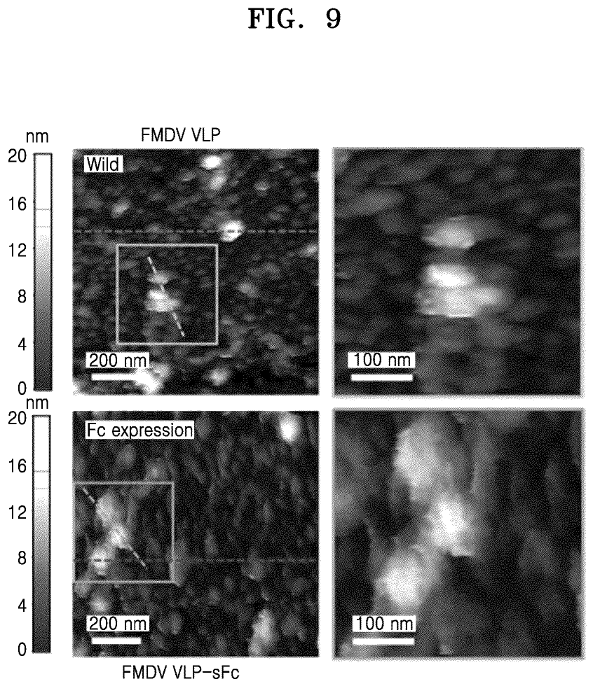

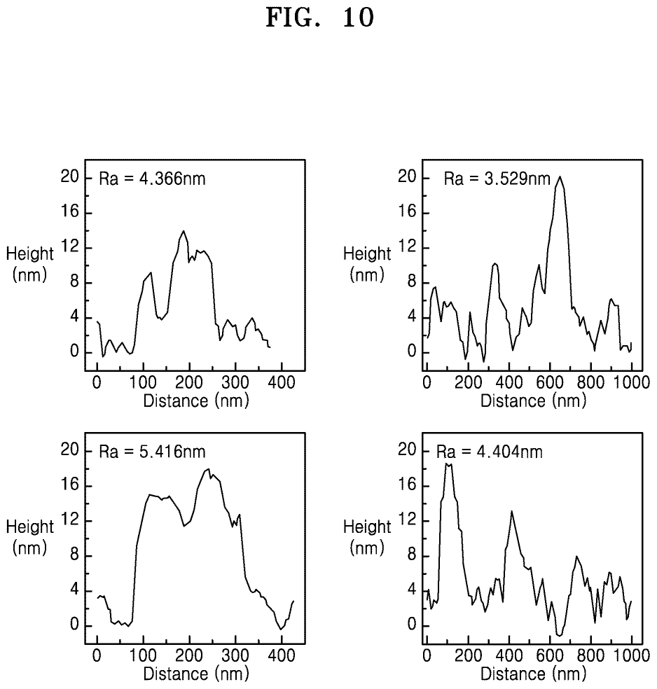

Specifically, virus-like particles generated from the FMDV VLP and FMDV VLP-sFc recombinant proteins prepared in Example 2-2 were observed by AFM. As a result, as shown in FIG. 9 and FIG. 10, it was confirmed that FMDV VLP particles were produced with a diameter of 32 to 60.2 nm and a height of 6.2 to 12.4 nm, and FMDV VLP-sFc particles were produced with a diameter of 47.8 to 67.2 nm and a height of 4.7 to 14.4 nm. Additionally, a roughness average (Ra) was analyzed using AFM software (xei), and the values were 3.53 to 4.34 nm for FMDV VLP and 4.40 to 5.42 nm for FMDV VLP-sFc. These results indicate that sFc was successfully attached to a VLP surface.

### Example 5: Confirmation of sFc-Tagged Virus-Like Particles Using Transmission Electron Microscopy (TEM) Technique

A FMDV VLP and FMDV VLP-sFc recombinant proteins prepared in Example 2-2 above were observed using a TEM Technique.

Specifically, for negative-staining EM, virus-like particles generated from the recombinant proteins prepared in Example 2 were diluted 2-fold with sodium phosphate buffer, and a suspension was applied to a formvar/carbon-coated grid (200 mesh) (Sigma Aldrich, TEM-FCF200CU50) for 3 minutes and then stained with 2% uranyl acetate. After removing excess uranyl acetate with filter paper, the grid was observed using TEM (ThermoFisher, Tecnai G2) at 120 kV.

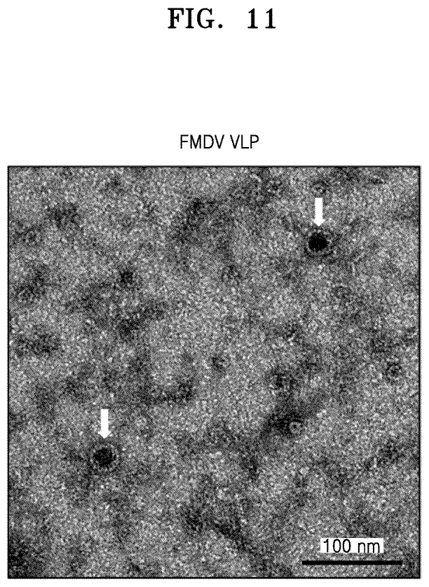

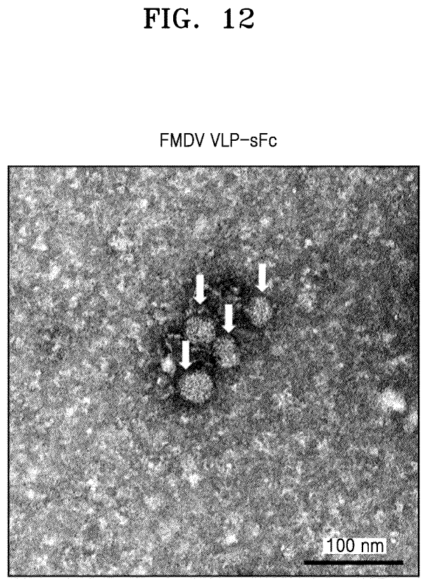

As a result, it was demonstrated that FMDV VLP had a size of about 30 nm and was consisted of a black protein mass in the center, and it was confirmed that FMDV VLP-sFc produced particles of about 40 nm in size (FIG. 11 and FIG. 12). These results indicate that sFc was successfully attached to a VLP surface. In particular, it was confirmed that FMDV VLP-sFc has a protrusion shape on the surface that is not present in FMDV VLP, confirming that sFc is expressed on the VLP surface.

### Example 6: Production and Confirmation of Recombinant Protein Using Adenovirus Vector

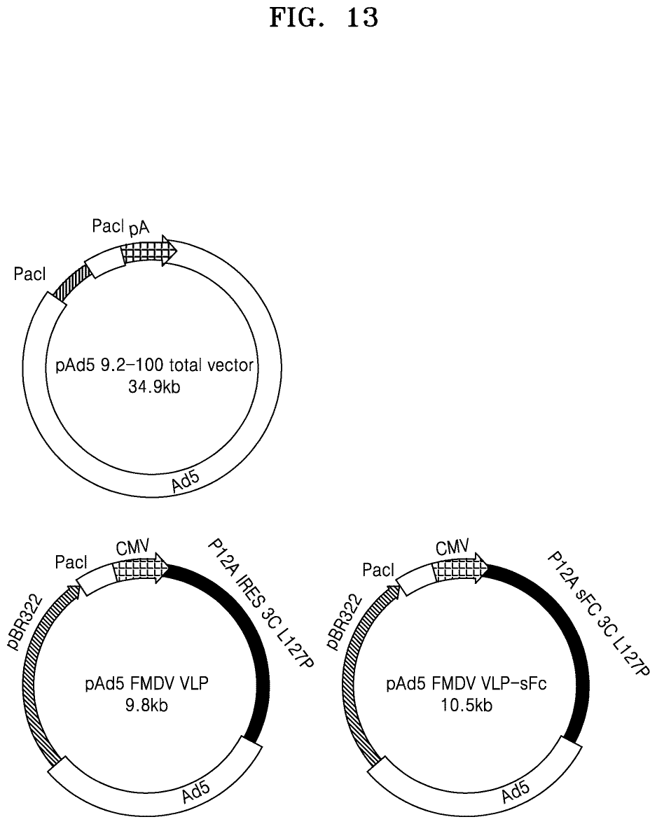

In the present example, a recombinant protein was produced using an adenovirus vector, and its immunogenicity was evaluated. Expression cassettes including a polynucleotide sequences synthesized in Example 1 were cloned using replication-deficient recombinant adenovirus vectors pacAd5 9.2-100 backbone vector and pacAd5 CMVK-NpA shuttle vector (Cell biolabs) according to the manufacturer's instructions, and recombinant expression vectors including genes shown in Table 4 below were acquired. A schematic diagram of the backbone vector used and the acquired recombinant expression vector is shown in FIG. 13.

TABLE 4

| Vector Name | Vector Information |
|---|---|
| pacAd5 9.2-100 backbone vector | Adenovirus backbone vector used to |
| (Cell biolabs) | produce recombinant adenovirus |
| | vector |
| pacAd5 CMVK-NpA shuttle vector | Adenovirus shuttle vector used to |
| (Cell biolabs) | produce recombinant adenovirus |
| | vector |
| pacAd5 CMVK-Npa FMDV VLP | A vector containing a P12A IRES 3C |
| | protease polynucleotide sequence |
| pacAd5 CMVK-Npa FMDV VLP- | A vector containing a P12A-sFc |
| sFc | IRES 3C protease polynucleotide |
| | sequence |

The recombinant expression vector was transfected into HEK293A (Thermo Fisher Scientific, R70507) cells according to the manufacturer's instructions to proliferate the recombinant adenovirus, which was then purified, dissolved in storage buffer solution [10 mM Tris-HCl (pH 80), 4% sucrose, 2 mM MgCl₂], and stored at −80° C. Afterwards, the purified adenovirus was transfected into A549 cells (KCLB, 10185) for 72 hours according to the manufacturer's instructions. Western blotting was conducted using the acquired A549 cells in the same method as in Example 22.

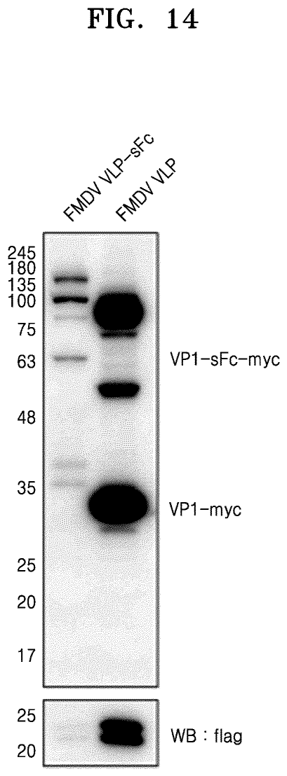

As a result, as shown in FIG. 14, an uncleaved recombinant protein P12A-sFc (120 kDa) and a cleaved VP1-SFc (63 kDa) were detected in lane 1. Additionally, uncleaved P12A precursor (85 kD) and cleaved VP1 (28 kDa) were detected in lane 2. These results indicate that when a pAd5 FMDV VLP-sFc vector according to an embodiment is used, FMDV VLP-sFc recombinant protein may be successfully formed and a cleaved VP1-sFc structural unit may be generated.

### Example 7: Immunogenicity Evaluation of FMD Virus-Like Particle Recombinant Proteins Produced Using Adenovirus Vector

In the present example, an immunogenicity of a vaccine composition including a recombinant protein according to an embodiment was evaluated, and for this purpose, the following experiment was conducted.

### (7.1) Intra-Animal Injection of FMDV VLP and FMDV VLP-sFc Recombinant Protein

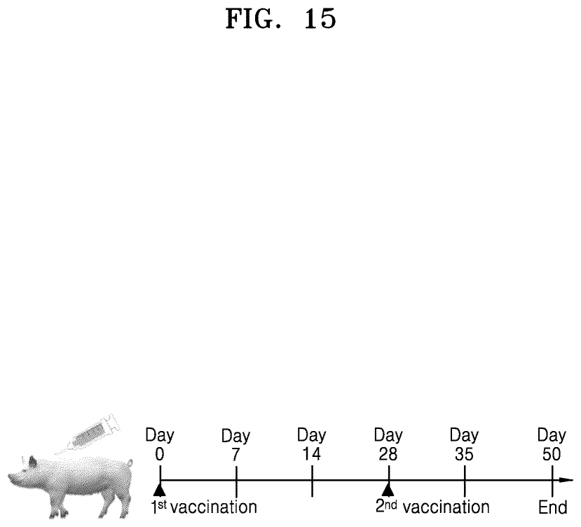

In the present example, in order to evaluate an immunogenicity of a vaccine composition including a recombinant protein prepared from Example 6, a vaccine composition was injected into pigs according to a schedule shown in FIG. 15, and then the expression levels of antibodies and cytokines were measured. In the present example, 16 six-week-old pigs were used and divided into 4 groups for injection as shown in Table 5. A group administered PBS was used as a negative control group. Additionally, a group administered with a commercial vaccine (BIOAFTOGEN® Biogenesis Bago) was used as a control group. All animal experiments were approved by the Chungnam National University Animal Experiment Ethics Committee.

TABLE 5

| | Number of | | | |
|---|---|---|---|---|
| | Experimental | | | Route of |
| Group | Pigs | Dosage | | Administration |
| Negative Control | 4 | 1 | ml | Intramuscular |
| Group | | | | Injection |
| Commercial vaccine | 4 | 1 | ml | Intramuscular |
| (BIOAFTOGEN ® | | | | Injection |
| Biogenesis Bago) | | | | |
| Ad5 FMDV VLP | 4 | 2 × 10⁸ | PFU/mL | Intramuscular |
| | | | | Injection |
| Ad5 FMDV | 4 | 2 × 10⁸ | PFU/mL | Intramuscular |
| VLP-sFc | | | | Injection |

Blood samples were collected from all experimental groups at intervals of 0, 7, 14, 28, 35, and 50 days after administration, and 50 days after the first vaccination, serum was separated from each group, and an enzyme-linked immunosorbent (ELLSA) assay and serum neutralization (SN) test were conducted.

### (7.2) Measurement of Neutralizing Activity Through Serum Neutralization Test

To measure the neutralizing activity following administration of a vaccine composition according to an embodiment, a serum neutralization test (SN test) was conducted. Specifically, a serum of each experimental group acquired from Example 7.1 was inactivated at 56° C. for 30 minutes and then serially diluted 2-fold. 100 TCID50/0.1 ml of FMDV was mixed with an equal amount of diluted serum at 37° C. for 1 hour. LF-BK cells were treated with 0.1 ml of each virus-serum mixture. After incubation at 37° C. for 1 hour, the cells were washed three times with PBS and maintained in DMEM at 37° C. for 3 days. A SN titer was expressed as a reciprocal of the highest serum dilution to indicate inhibition of cytotoxicity. The individual neutralizing activities of all sera collected from vaccinated subjects were evaluated against a FMDV 01-Manisa strain (virus neutralization test, VNT).

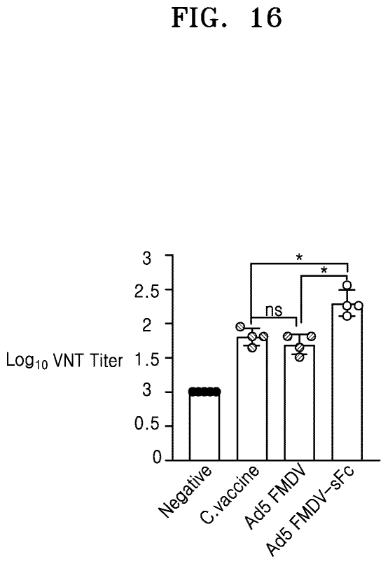

As a result, as shown in FIG. 16, no neutralizing activity was observed in the negative control group, the SN titer of a commercial vaccine group ranged from 1.81 to 1.95 (average 1.8), and the SN titer of an Ad5 FMDV VLP vaccine group ranged from 1.65 to 1.81 (average 1.67). The SN titers in the Ad5 FMDV VLP-sFc vaccine group ranged from 2.11 to 2.56 (average 2.22). These results confirmed that a vaccine composition including a recombinant protein according to an embodiment may induce a significantly superior level of neutralizing activity.

### (7.3) Measurement of Cytokine Expression Levels Through ELISA Analysis

In order to confirm the level of immune activity induction of a vaccine composition including a recombinant protein according to an embodiment, an expression level of immune-related factors, interferon gamma (IFN-γ), interleukin-12 (IL-12), TNF and IL-4, in a serum acquired from Example 7.1 was confirmed through ELISA analysis.

A sandwich ELISA assay method was used, and the serum from each experimental group was analyzed for IFN-γ (Cusabo, CSB-E06794p), IL-12 (Cusabo, CSB-E11341p), TNF (Cusabo, CSB-E16980p), and IL-4 (Cusabo, CSB-E06785p) following the manufacturer's instructions for the Cusabio ELISA kits.

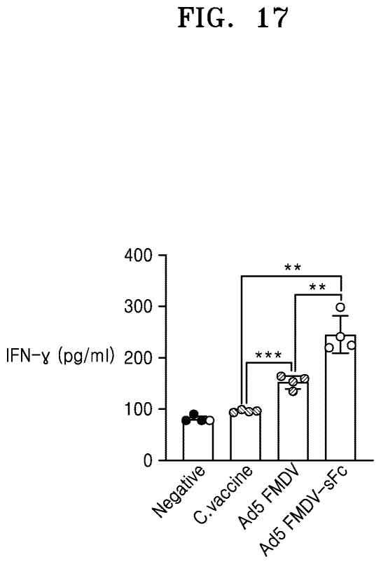

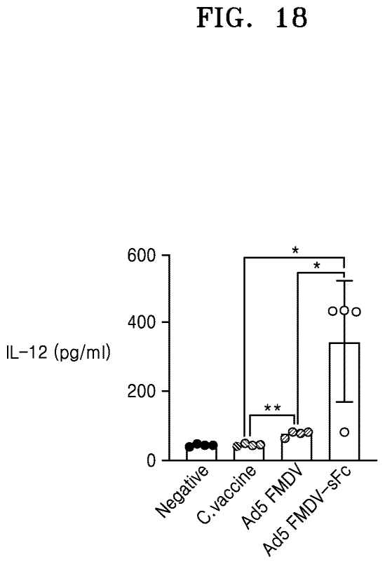

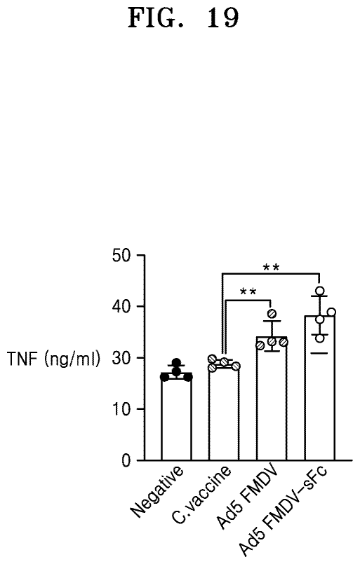

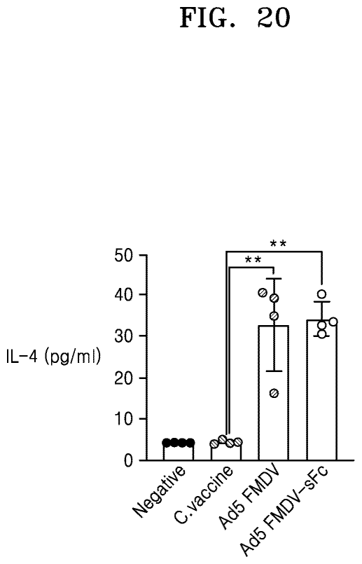

As a result, it was confirmed that a Ad5 FMDV VLP-sFc vaccination group significantly increased the production of IFN-γ, IL-12, TNF, and IL-4 to superior levels (FIG. 17 to FIG. 20).

These results indicate that a vaccine composition including a recombinant protein according to an embodiment has excellent vaccine efficacy by inducing expression of immune-related factors at a significantly superior level.

The foregoing description of the present disclosure is for illustrative purposes only, and one that has ordinary skill in the art to which the present disclosure belongs will understand that the present disclosure may be readily adapted to other specific forms without altering the technical ideas or essential features of the present disclosure. Therefore, the examples described above should be understood in all respects as illustrative and not restrictive.

1. A recombinant protein comprising a P12A protein derived from a foot and mouth disease virus (FMDV) capsid and a fragment crystallizable (Fc) region of a swine-derived immunoglobulin.

2. The recombinant protein of claim 1, wherein the P12A protein derived from the FMDV capsid consists of an amino acid sequence of SEQ ID NO: 1.

3. The recombinant protein of claim 1, wherein the Fc region of the swine-derived immunoglobulin is directly linked or fused to the P12A protein derived from the FMDV capsid, or is linked to the P12A protein derived from the FMDV capsid by a linker.

4. The recombinant protein of claim 1, wherein the recombinant protein further comprises a 3C protease protein consisting of an amino acid sequence of SEQ ID NO: 2.

5. The recombinant protein of claim 1, wherein the recombinant protein self-assembles within a host cell to form a virus-like particle.

6. A polynucleotide encoding the recombinant protein according to claim 1.

7. A vaccine composition for preventing or treating a foot and mouth disease virus (FMDV) infectious disease, comprising the recombinant protein of claim 1 as an active ingredient.

8. The vaccine composition of claim 7, wherein the vaccine composition is selected from the group consisting of compositions for intramuscular administration, subcutaneous administration, intraperitoneal administration, intravenous administration, dermal administration, ocular administration, and brain administration.

9. The vaccine composition of claim 7, wherein the vaccine composition further comprises a pharmaceutically acceptable excipient, diluent or carrier.

10. A method of preventing or treating a foot and mouth disease virus (FMDV) infectious disease, comprising administering the vaccine composition of claim 7 to a subject other than a human.
